(19) United States
(12) Patent Application Publication
(10) Pub. No.: US 2015/0361503 A1
(43) Pub. Date: Dec. 17, 2015

(54) METHODS FOR SELECTING THERAPEUTICS FOR TREATMENT OF HER2+ CANCERS

(71) Applicant: Merrimack Pharmaceuticals, Inc., Cambridge, MA (US)

(72) Inventors: Jinyan DU, Cambridge, MA (US); Daniel C. KIROUAC, Cambridge, MA (US); Maria J. LAHDENRANTA, Cambridge, MA (US)

(21) Appl. No.: 14/737,301

(22) Filed: Jun. 11, 2015

Related U.S. Application Data
(60) Provisional application No. 62/013,366, filed on Jun. 17, 2014.

(51) Int. Cl. C12Q 1/68; A61K 45/06; A61K 39/395

(52) U.S. Cl. CPC C12Q 1/6886; A61K 39/3955; A61K 45/06; A61K 2039/505

(57) ABSTRACT

Described are methods of selecting appropriate therapy for, or optimizing therapeutic efficacy of treatment of, a patient having a cancer using biomarker readings obtained from a biological sample of the cancer from the patient and, based on the readings obtained, treating the patient with MM-111 in combination with trastuzumab and one of the following: a receptor tyrosine kinase inhibitor, a mitogen-activated protein kinase kinase (MEK) inhibitor, and a protein kinase B (AKT) inhibitor.

### BACKGROUND

Overexpression of the receptor tyrosine kinase HER2, via focal amplification of ERBB2 occurs in approximately 20% of breast and gastric cancers, and at lower frequencies in many other solid tumors. Understanding the molecular pathways by which the HER2 drives cancerous cell growth is critical to the design of improved treatment strategies. Overexpression of this receptor is known to drive ligand-independent receptor homo-dimerization and receptor-mediated signaling. HER2 and other receptor tyrosine kinases (RTKs) are reported to mediate cellular effects primarily through regulating activity of the phosphoinositol-3-kinase (PI3K) cascade. Mutational activation of this cascade (via PIK3CA point mutations, or PTEN deletions) is known to mediate resistance to HER2-targeted therapies trastuzumab and lapatinib from both pre-clinical models and through retrospective analysis of clinical data. Many small molecule inhibitors of components of the PI3K cascades are in clinical development, targeting PI3K, AKT, and mTOR. However, clinical experience to date with these drugs has revealed, rather unexpectedly, that mutational status of the pathway components (PIK3CA or PTEN) is not predictive of patient responses.

The MAPK/ERK signaling cascade is another well-known driver of oncogenic cell growth, and small molecule inhibitors targeting the MAPK/ERK pathway kinase MEK are in clinical development for in a variety of solid tumors. While critical for transducing signals emanating from the BRAF and KRAS oncogenes, the MAPK/ERK pathway is not believed to play a critical role in mediating HER2 signaling. Unfortunately, attempts at dual inhibition for the PI3K and MAPK cascades thus far have revealed that such combinations can be extremely toxic.

Improved predictive biomarkers of functional pathway dependence are therefore needed to allow selection of effective therapeutic agents. Disclosed herein are methods designed to address this need.

### BRIEF DESCRIPTION OF THE DRAWINGS

The application file contains at least one drawing executed in color. Copies of this patent or patent application with color drawings will be provided by the Office upon request and payment of the necessary fee.

FIGS. 1A-1C show that HER2+ cancer models can be classified by PI3K vs. MAPK pathway dependence, and that pathway dependence correlates with genetic and cellular properties. FIG. 1A shows the relative PI3K/AKT vs. MAPK/ERK dependence (Pathway Bias) across 18 HER2+ cell lines in the presence of exogenous heregulin (“HRG”) stimulation or its absence (“FBS”). The tissue types from which the tumor cell lines are derived (breast, lung, ovarian, and stomach/esophageal) is indicated in the bar below the graph. FIG. 1B shows the genetic status of PI3K-pathway components (black dots indicated mutant status and white dots indicate wild-type, top panel-“Genetics”) and in vitro proliferation rates for each cell line as shown by cell population density at 96 hours (pale dots indicate FBS only, dark dots indicate HRG stimulation, bottom panel-“PD”). FIG. 1C shows representative response surfaces for AKT and MEK inhibitor combinations in the absence (top panels) and presence (bottom panels) of exogenous HRG for PI3K-dependent, MAPK-dependent, and switching class cells.

FIGS. 2A-2C show that multivariate protein biomarkers predict pathway dependence and sensitivity to multiple anti-cancer agents. Red indicates positive Log10IC50 values and blue indicates negative Log10IC50 values (PI3K dependence). FIG. 2A is a graph showing that rank correlation coefficients between protein expression and four model parameters, hierarchically clustered by Pearson's correlations. FIG. 2B shows a logistic regression-based prediction of pathway Bias based on protein expression. FIG. 2C shows relative IC50 values of cancer drugs from the Genomics of Drug Sensitivity in Cancer database between groups of predicted PI3K vs. MAPK dependent HER2+ cells based on expression of ErbB3 and EGFR.

FIGS. 3A-3D show that triple targeting of the HER2/HERS complex with MM-111, lapatinib and trastuzumab is predicted to be broadly effective in both PI3K and MAPK-dependent cells, but resistance to the combination is predicted to be mediated by activating mutations in the respective intracellular signaling pathways. FIG. 3A shows the results of in silico screening of relative tumor growth in PI3K-dependent cells in response to 32 combinations of five drugs (black=present, white=absent from regimen). FIG. 3B shows the results of in silico screening of relative tumor growth in MAPK-dependent cells in response to 32 combinations of five drugs (black=present, white=absent from regimen). FIG. 3C shows the predicted relative growth of PI3K-mutant (“PI3K mut”, white bars), MAPK-mutant (“RAF mut”, black bars), and wild type cells (dark and light gray bars) to indicated combinations of the three drugs, along with an AKT inhibitor and a MEK inhibitor. FIG. 3D shows the actual frequency of HER2 amplification associated with PI3K vs. MAPK activating mutations in primary tumor resections (tumor types as indicated).

FIGS. 4A-4C show that resistance of PI3K and/or MAPK activating mutations to lapatinib-containing combination therapy regimens can be overcome by substituting an AKT inhibitor or MEK inhibitor. FIG. 4A is a graph showing the in vitro proliferative responses (% growth inhibition at 100 nM) to select drug combinations across eight cell lines (right vertical axis), with and without stimulation by exogenous heregulin. Cell lines are classified by mutational status of the PI3K (PIK3CA, PIK3R1, PTEN) and MAPK pathways (KRAS), and hierarchically clustered by response patterns. White indicates wild-type, black indicates activating mutations, and stippled indicates non-activating PI3K mutations (left vertical axis). FIG. 4B shows the average regression coefficients (β) for each drug across the 3 genetic cell line classes, +/− heregulin. FIG. 4C is a drawing of a decision tree based on the above results. This decision tree shows the application of the invention to the selection of therapeutic agents depending upon tumor biomarker profiles.

### DETAILED DESCRIPTION

Combinations of targeted therapies are currently undergoing clinical evaluation for treating trastuzumab-refractory HER2+ disease. However, the molecular and genetic determinants of sensitivity to the combinations remain obscure. Rational strategies to predict combination regimen activity based on molecular features of patients' tumors would thus be highly valuable. The study described herein (see Examples, below) was designed to characterize the molecular need for functional diversity of HER2+ cancer via systematic analysis of 20 HER2+ cancer cell lines, to quantitatively assess how dependence upon the canonical PI3K/AKT and MAPK/ERK cascades varies, whether pathway dependence could be predicted from proteomic and genomic biomarkers, and whether such molecular and functional features could be utilized to design personalized combinations of therapeutics.

### EXAMPLES

### Materials and Methods

### Cell Lines and Reagents

AU565, HCC1419, NCI-H2170, HCC202, HCC1954, NCI-N87, ZR75-1, SKOV3, ZR75-30, MDAMB175VII, CALU3, MDAMB453, MDAMB361, JIMT1, SKBR3 and HCC2218 cells were obtained from ATCC. OE19 and OE33 were obtained from ECCC, COLO-678 is obtained from DSMZ, and KYSE-410 from Sigma-Aldrich.

NCI-N87 PIK3CA-mutants are generated by transducing NCI-N87 cells with full-length PIK3CA-H1047R mutant (GeneCopoeia, Inc.) expressing lentivirus (also encoding PAC-PA-turboGFP for selection). A polyclonal line was established after selecting for puromycin and sorting for GFP+ cells. A control cell line (NCI-N87 GFP) was engineered to express EGFP alone in the same manner. All cell lines are maintained in RPMI supplemented with 10% FBS, penicillin, and streptomycin. MM-111 was produced in-house at Merrimack Pharmaceuticals, trastuzumab (Genentech/Roche) was obtained from a hospital pharmacy, lapatinib was purchased from LC Laboratories, and AZD6244, BKM-120, GDC-0941, trametinib (GSK-1120212), MK-2206, PD0325901 and triciribine were purchased from Selleck Biochem. Recombinant human HRG-β1 (EGF domain) was purchased from R&D Systems. The amino acid sequence of MM-111 is disclosed in U.S. Pat. No. 8,927,694, incorporated herein by reference.

### In Vitro Cell Growth Assays: AKT & MEK Inhibitor Responses

Cells are seeded at 600 cells per 384-well plate in 4% FBS cell growth medium, stimulated (or not) with 2 nM HRG-β1 for four hours, and then treated with individual or combinations of the AKT and MEK inhibitors. In vitro proliferation was tracked over five days in culture by video microscopy (IncuCyte®, Essen BioScience).

### In Vitro Cell Growth Assays: Drug Combination Effects

Combination effects between MM-111, trastuzumab, lapatinib, MK-2206 and trametinib are evaluated by CTG cell viability assays. Cells (BT474-M3, NCI-N87, NCI-N87-PIK3CA(wt), NCI-N87-PIK3CA(H1047R), NCI-N87-PIK3CA(E454K), COLO-678, KYSE-410, ZR75-1, MDA-MB-361 or MDA-MB-175-VII) are seeded at 700 cells per 384-well plate in 10% FBS cell growth medium and treated with the five drugs, separately or in combination, at 1, 0.1, 0.01 and 0.01 μM, with and without 5 nM HRG-β1 pre-stimulation (for four hours). Cell viability was determined 72 hours later with CellTiter-Glo® luminescent cell viability assay (Promega).

### Cellular Protein Lysate Preparation

Cell lines are seeded at 7,500 cells per well in 384-well culture plates in RPMI containing 4% FBS. 48-hour post plating, cells were stimulated (or not) with 2 nM HRG-β1. Four hours post-stimulation, AKT or MEK inhibitors were added at concentrations indicated. Total protein lysates were harvested at 1, 4, 24-hour post drug treatment. At harvest, cells were placed on ice, and 70 μl RIPA lysis buffer (Sigma-Aldrich) supplemented protease inhibitor and phosphatase inhibitor tablets (Roche) was added to each well. The plates were stored at −80° C. until analysis. On the first day of protein profiling, the lysates were thawed at 4° C. and centrifuged at 4000 rpm for 10 minutes. The supernatant was used for further analysis with multiplex Luminex protein assays as described below.

### Multiplex (Luminex) Protein Assays

Antibodies (see Table 1) are conjugated to MagPlex® beads (Luminex Corp.) by incubating 20 micrograms of antibody with beads according to the manufacturer's instructions. Conjugated beads are then mixed and diluted 1000-fold in phosphate buffered saline (PBS)-1% bovine serum albumin (BSA) (Sigma). Diluted beads are transferred into 384-well assay plates (Corning) at 30 μl per well and then washed three times with PBS-1% BSA. Washed beads are incubated with 20 μl of total protein lysates overnight with shaking at 4° C. The beads are then washed with PBS-1% BSA. Detection antibodies (Table 2) are added and incubated at 4° C. overnight with shaking. After washing with PBS-1% BSA, streptavidin-conjugated phycoerythrin (Invitrogen) was added at 2 μg/ml and incubated at room temperature for 30 min. Finally, the beads are washed with PBS-1% BSA. Data were acquired with a FlexMap3D® instrument (Luminex Corp.) according to the manufacturer's instructions. Antibodies are listed in Table 1 and Table 2.

TABLE 1
Capture antibodies used in Luminex assays

| Target | Total/phospho | Source | Cat. No. |
|---|---|---|---|
| AKT | total | RnD | DYC887B |
| AKT | pT308 | CST | 4056BF |
| AKT1 | pS473 | Millipore | 05-669 |
| AXL | total | RnD | MAB154 |
| CDKN1B | total | RnD | DYC2256E |
| EGFR | total | Thermo | MS-609 |
| EGFR | total | Thermo | MS-396 |
| EPHA2 | total | RnD | MAB3035 |
| ERBB2 | total | BioLegend | 324402 |
| ERBB2 | total | RnD | MAB1129 |
| ERBB2 | total | RnD | DYC1768E |
| ERBB3 | total | RnD | MAB3481 |
| ERBB3 | total | RnD | DYC1769E |
| ERBB4 | total | Thermo | MS-270 |
| ERBB4 | total | RnD | DYC2115E |
| ERK1/2 | pT202Y204 | CST | 4370BF |
| ERK1/2 | total | CST | 3374BF |
| FGFR1 | total | RnD | MAB658 |
| FGFR2 | total | RnD | MAB6841 |
| FGFR3 | total | RnD | MAB766 |
| FGFR3 | total | RnD | MAB7661 |
| FGFR4 | total | RnD | MAB685 |
| IGF1R | total | RnD | MAB391 |
| INSR | total | RnD | MAB1544 |
| MERTK | total | RnD | DYC2579 |
| MET | total | RnD | MAB3581 |
| RPS6 | pS235S236 | CST | 2211BF |
| RPS6 | pS240S244 | CST | 2215BF |
| RPS6 | pS235S236 | CST | 4858BF |
| SRC | total | RnD | DYC2685 |

TABLE 2
Detection antibodies used in Luminex assays

| Target | Total/phospho | Source | Cat. No. |
|---|---|---|---|
| ERK1/2 | total | CST | 4695BF |
| EGFR | total | CST | 6627 |
| ERBB2 | total | RnD | BAF1129 |
| ERBB3 | total | RnD | BAM348 |
| ERBB4 | total | RnD | BAF1131 |
| AKT | total | RnD | DYC1775 |
| CDKN1B | total | RnD | DYC2256 |
| Tyrosine | phospho | Millipore | 16-103 |
| RPS6 | phospho | RnD | DYC3918 |

### Example 1

### Computational Model Design

### Logic-Based Models of Cell Growth Regulation

Observed changes in cell density over time are determined by the balance of cell proliferation vs. death within the culture. Both cell proliferation and survival are regulated by PI3K/AKT and MAPK/ERK signaling cascades, which assuming an exponential growth can be expressed as:

$$\frac{d X}{d t} = {μ}_{MAX} \cdot {f}_{1} ( pAKT , pERK ) - {δ}_{MAX} \cdot {f}_{2} ( pAKT , pERK )$$

Where X=number of cells (assumed proportional to surface area), μMAX=maximum rate of proliferation, δMAX=maximal rate of cell death, and f1 and f2 are functions integrating pAKT and pERK signaling.

A quantitative logic-based formalism was developed to describe changes in cell density as function of PI3K/AKT and MAPK/ERK pathway activation. AKT and MEK inhibitor concentrations (μM) are used as surrogates for pathway activities, assuming monotonic dose-response relationships. As the logic by which cells integrate and interpret these signals remains obscure, nine alternate growth regulatory functions were initially assessed, combining null (K), OR, and AND-type logic gates as proliferation and survival functions (f1 and f2):

$$K = 1$$

$$OR = \frac{{( {w}_{akt} \cdot AKT + {w}_{erk} \cdot ERK )}^{k}}{τ + {( {w}_{akt} \cdot AKT + {w}_{erk} \cdot ERK )}^{k}}$$

$$AND = ( \frac{{AKT}^{{k}_{—} akt}}{{τ}_{akt} + {AKT}^{{k}_{—} akt}} ) \cdot ( \frac{{ERK}^{{k}_{—} erk}}{{τ}_{erk} + {ERK}^{{k}_{—} erk}} )$$

TABLE 3
Model Parameters

| | MODEL | | | | | | | | |
|---|---|---|---|---|---|---|---|---|---|
| | M1 | M2 | M3 | M4 | M5 | M6 | M7 | M8 | M9 |
| PROLIF | K | OR | AND | K | K | OR | OR | AND | AND |
| DEATH | K | K | K | OR | AND | OR | AND | OR | AND |
| PARAMS | 2 | 6 | 6 | 6 | 6 | 10 | 10 | 10 | 10 |

Parameters for each of the nine models were estimated for each cells line using a Particle Swarm Optimization algorithm (ladevaia, Lu, Morales, Mills, & Ram, 2010) minimizing the mean squared error between experimental measurements (fold cell expansion over 96 hours) and model simulations. Relative model performance was assessed using the Akakie Information Criterion (AIC):

AIC=2·P+N·log₁₀(MSE)

Where P=number of parameters (2-10), N=number of experimental measurements (30), and MSE=mean squared error.

The fourth model structure assessed (M4), consisting of an OR-Gate regulating cell survival, was found to be optimal for the largest number of cell lines tested. The final formulation of the cell growth regulatory model used in subsequent analyses was thus:

$$\frac{d X}{d t} = {μ}_{MAX} - {δ}_{MAX} ( \frac{{( {w}_{akt} \cdot {AKT}_{i} + {w}_{erk} \cdot MEKi )}^{k}}{τ + {( {w}_{akt} \cdot AKTi + {w}_{erk} \cdot MEKi )}^{k}} )$$

Pathway Bias was then defined as the normalize differential between the parameters wakt and werk:

$$Bias = \frac{( {w}_{akt} - {w}_{erk} )}{( {w}_{akt} + {w}_{erk} )}$$

Thus a Bias of 1 indicates complete dependence upon PI3K/AKT signaling, −1 MAPK/ERK signaling, and 0 as balanced between the two cascades.

### Logistic Regression Model of Pathway Bias

The Pathway Bias measurement for each cell was first discretized into MAPK vs. PI3K-dependence (Bias=−1 vs. +1) given the bimodal distribution of this metric. The probability of MAPK-dependence (PMAPK) vs. PI3K-dependence (PPI3K=1−PMAPK) was then modeled as a function of input features (protein signals, genetic status, proliferation rate, and tissue-type) using a logistic regression equation:

$$ln ( \frac{{P}_{MAPK}}{{P}_{PI 3 K}} ) = {β}_{0} + {\sum }_{i = 1}^{N} {β}_{i} \cdot {X}_{i}$$

Where N=number of features (Xi) and βi=regression coefficients. The βi parameters were estimated by maximum likelihood estimation, and predictive power of the model assessed using leave-one-out cross validation (LOOCV) procedure. Model-predicted Bias was then back-calculated using the probabilities as:

Predicted Bias=−1·P_{MAPK}+1·P_{PI3K}

### Translational Model and in Silico Drug Combination Screening

The semi-mechanistic model connecting ErbB receptor signaling, through PI3K/AKT and MAPK/ERK cascades, to tumor growth is described below, and in Kirouac et al., Science Signaling (2013) Iss 288, v6. Quantitative logic-based equations were used to describe phosphorylation status of HER2 and HERS receptors as functions of heregulin, MM-111, and lapatinib concentrations, and downstream pAKT and pERK status using OR-gates integrating phosho-HER2 and HERS levels. Cell surface expression of total HER2 and HERS receptors were described using control theory-based differential equations, where receptor expression is negatively regulated by pAKT and pERK. Cell growth is described using ODEs of the form shown above, with transient compartments included to account for time lag in signal propagation between drug exposure and phenotypic responses as described in Yang et al., The AAPS Journal, Vol. 12, No. 1, March 2010. PI3K/AKT pathway dependence was simulated by setting wakt=0.975 and werk=0.05, and conversely for MAPK/ERK pathway dependence. Tumor heterogeneity was simulated via Monte Carlo sampling of the following model parameters (prospective biomarkers) from log-uniform distributions (Table 4).

TABLE 4
Model Parameters

| Pi | variable | Description | LB | UB |
|---|---|---|---|---|
| P1 | HRG | exogenous HRG (M) | 10-12 | 10-9 |
| P15 | w6 | pE2 activation of PI3K | 0.01 | 0.25 |
| P16 | w7 | pE3 activation of PI3K | 0.5 | 1 |
| P18 | tau5 | pPI3K activation of pAKT; AND | 102 | 105 |
| P19 | w8 | pE2 activation of pMAPK | 0.5 | 1 |
| P20 | w9 | pE3 activation of pMAPK | 0.01 | 1 |
| P22 | tau7 | pMAPK activation of pERK | 3 × 102 | 3 × 105 |
| P36 | g_AKT | AKT feedback: gain (foldX) | 0.5 | 5 |
| P40 | g_ERK | ERK feedback: gain (foldX) | 0.5 | 5 |
| P44 | g_E2 | E2 feedback: gain (foldX) | 0.5 | 10 |
| P47 | E2_basal | basal E2 level (mol/cell) | 105 | 5 × 106 |
| P49 | g_E3 | E3/pAKT feedback: gain (foldX) | 0.5 | 10 |
| P52 | E3_basal | basal E3 level (mol/cell) | 104 | 5 × 105 |
| P53 | g_E3erk | E3/pERK feedback: gain (foldX) | 0.5 | 10 |

For drug screening, tumor growth was simulated over 2 week periods, with tyrosine kinase inhibitors (lapatinib, AKTi, and MEKi) administered daily, and biologics (MM-111 and trastuzumab) administered weekly.

### Multivariate Linear Regression Models of Drug Combination Effects

Cell growth inhibition (CGI) as measured by CellTiter Glow® (CTG) assay over the 96-hour in vitro culture [CTGCTRL−CTGTREAT)/CTGCTRL] was described using a multivariate linear regression function of the Log10 drug concentrations (Ci):

$$CGI = {\sum }_{i = 1}^{N} {β}_{i} \cdot {log}_{10} {C}_{i}$$

Where N=number of input drugs (5: MM-111, lapatinib, trastuzumab, MK-2206, and GSK-1120212), and βi=regression coefficients, estimated by maximum likelihood estimation.

### Example 2

### PI3K vs. MAPK Pathway Bias Varies across 18 HER2+ Cell Lines, and is Effected by HRG Stimulation

A panel of HER2+, but otherwise diverse, cell lines was assembled in order to examine how dependence on the PI3K and MAPK cascades varies across HER2+ cancers. This panel included breast, lung, gastrointestinal, and ovarian cancer cell lines. To characterize pathway dependence, each cell line was treated with a full 5×6 dose combination matrix of the AKT inhibitor (AKTi, MK2206) and MEK inhibitor (MEKi, trametinib) covering a 3-fold dilution series starting from 1 μM (AKTi) and 10 μM (MEKi). In vitro cell proliferation was then quantified via video microscopy over 96 hours in the presence or absence of the HERS ligand heregulin (HRG). To characterize the shapes of these cell growth surfaces, quantitative logic-based models of cell growth kinetics were parameterized for each cell line. These simple phenomenological models characterize the balance of cell proliferation vs. cell death as functions of drug concentration (and by extension, pathway activity) using combinations of quantitative OR and AND-gates. While nine alternate model variations were assessed, a simple logical OR-gate was found to perform optimally across the panel on average. The OR-gate model additionally has the benefit of ease of interpretation for parametric comparison between cells. Six parameters consist of the maximal proliferation rate and cell death rates (μmax, δmax), EC50 and Hill coefficients (τ, k), and empirical weights toward PI3K and MAPK dependence (w_akt, w_erk), as described in the Materials and Methods section above. To develop a single metric of pathway dependence for comparative analysis, Pathway Bias is defined herein as the normalized difference of the weighting parameters, where a value of 1 signifies complete PI3K-dependence, 0 PI3K and MAPK pathway dual-dependence, and −1 complete MAPK-dependence.

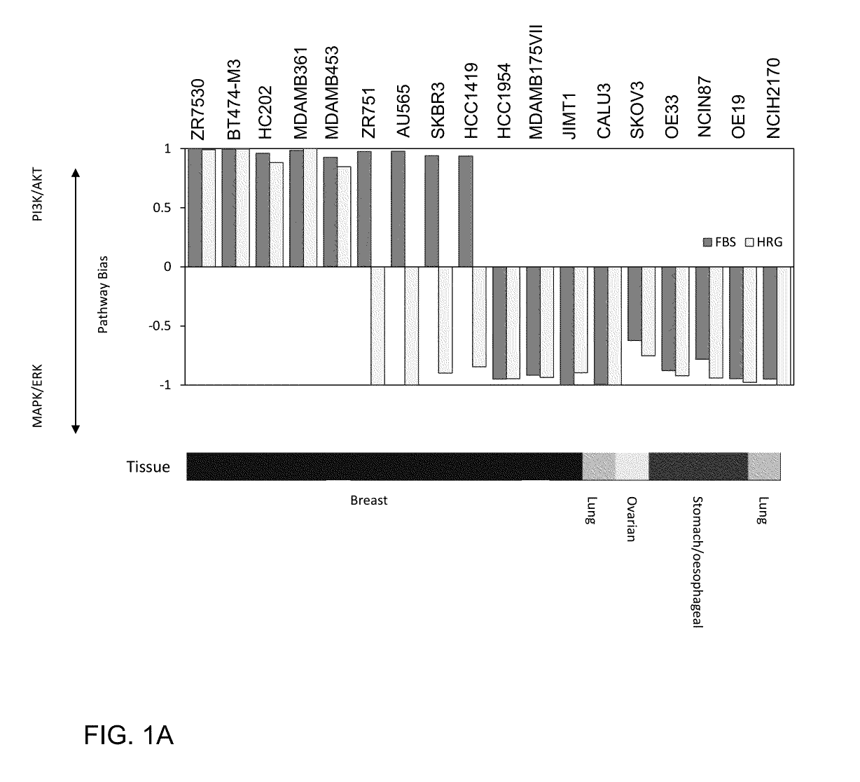

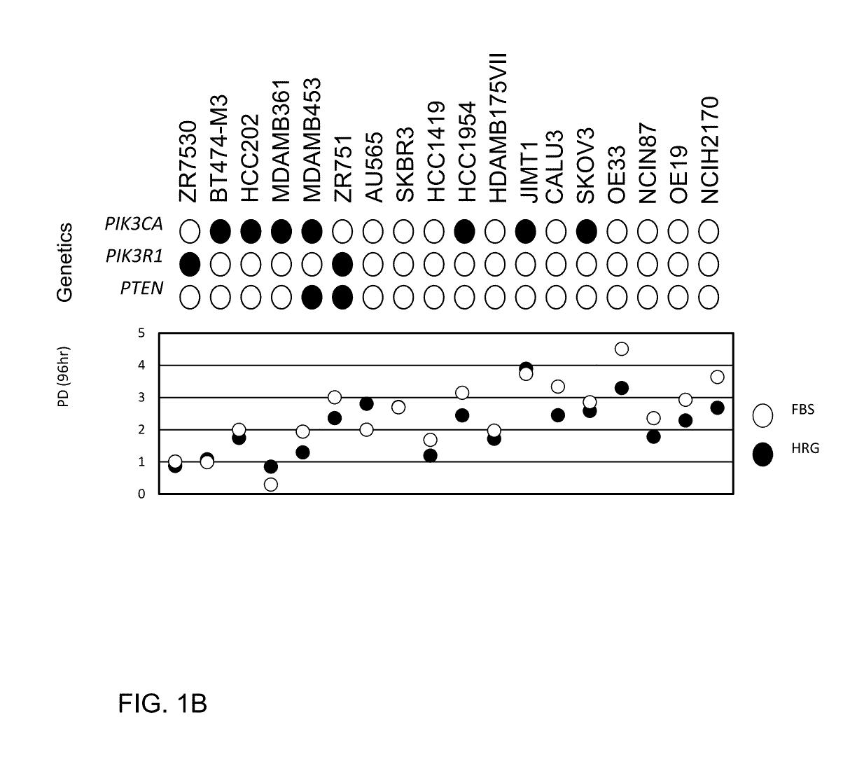

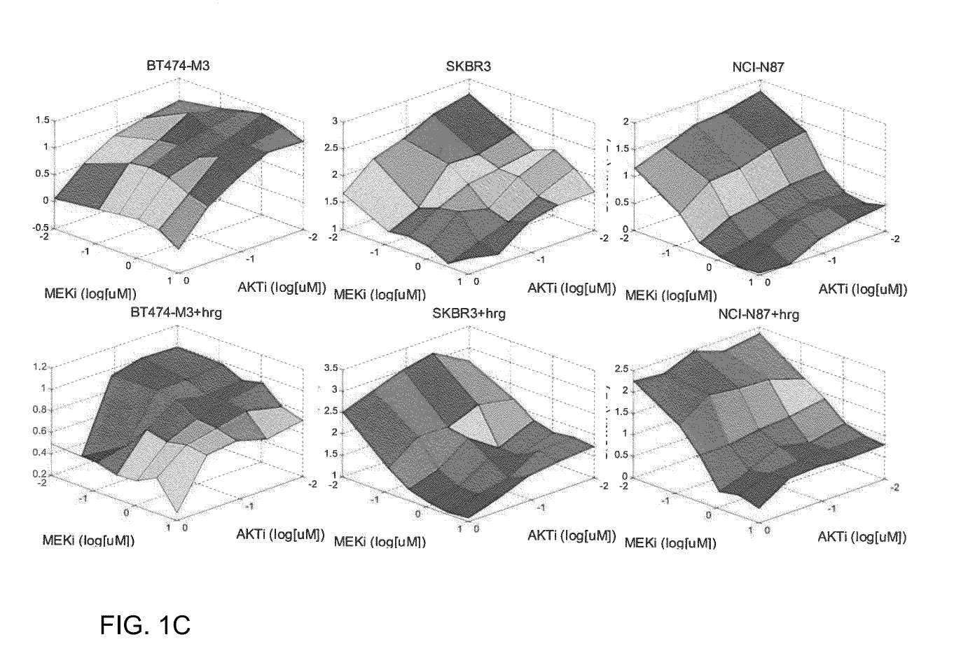

As shown in FIG. 1A, 5/20 cell lines could be classified as PI3K-dependent, 9/20 as MAPK-dependent, and unexpectedly 4/20 switch from PI3K to MAPK-dependence upon HRG stimulation. Heregulin stimulation resulted in all cells displaying some degree of reduced sensitivity to AKT inhibition and increased sensitivity to MEK inhibition, though the effect was much more pronounced in the Switching class. Overlaying information on tissue source, mutational profiles on select genes (from COSMIC and CCLE databases), and basal proliferation revealed some intuitive patterns. First, all of the non-breast indications were MAPK-dependent, whereas breast cancers covered all three functional classes.

While PI3K-biased cells are enriched for PIK3CA, PIK3R1, and PTEN mutations (as expected), mutational status was not a predictive classifier, as some MAPK-dependent cells harbored PIK3CA mutations (FIG. 1B, Genetics). These genetic metrics alone (HER2 status and PI3K pathway mutations) are thus insufficient for determining pathway dependence of the tumors, and by extension the relative sensitivity to PI3K/AKT and MEK targeted inhibitors. This is clinically important, given that PI3K/AKT inhibitors are currently being employed in clinical trials for HER2+ disease, either in unselected patient groups or stratified by PI3KCA/PTEN status alone.

FIG. 1C shows representative response surfaces for AKT and MEK inhibitor combinations in the absence (top panels) and presence (bottom panels) of exogenous HRG for PI3K-dependent, MAPK-dependent, and switching class cells.

### Example 3

### Expression Patterns of EGFR, HERS, and P27 Predict Pathway Bias and Drug Sensitivity

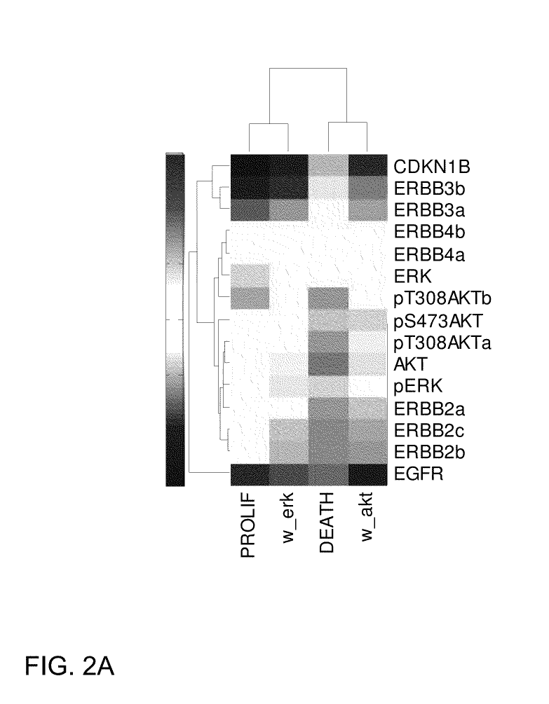

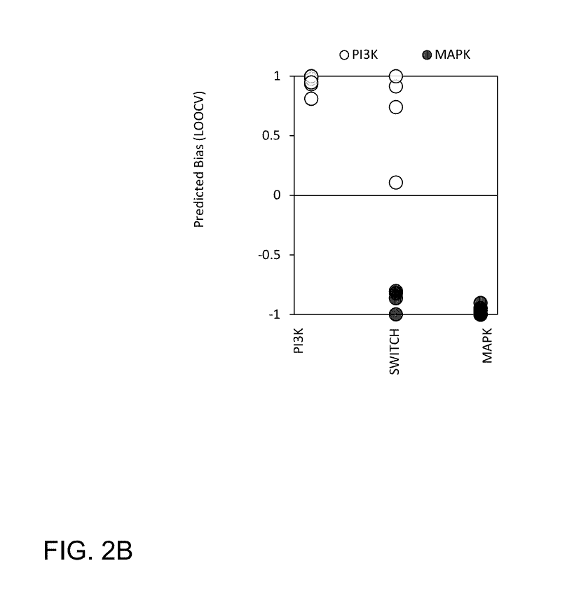

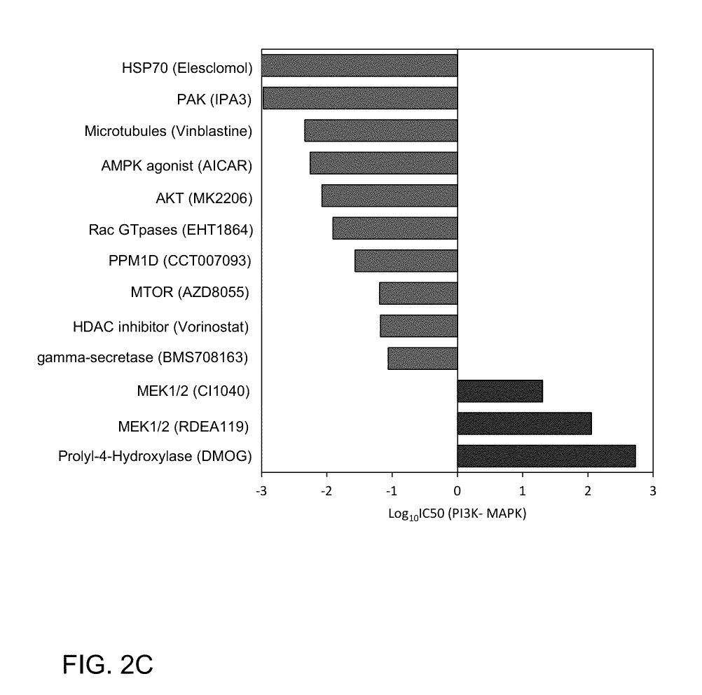

As described in Example 2, mutational status is insufficient to accurately predict PI3K or MAPK pathway dependence. A select panel of protein markers was therefore used to predict signaling pathway dependence and other phenotypic characteristics of the cells. The panel of cell lines described in Example 2 was profiled for ErbB receptor expression, total and phosphorylated forms of ERK and AKT, and the cell cycle regulator CDKN1B (p27) using quantitative Luminex assays. Rank correlation coefficients between each protein analyte and the characteristic model parameters were then computed across the cell line panel, represented as a hierarchically clustered heatmap (FIG. 2A). Functional relationships were revealed by this relatively simple modelling and analysis; highly proliferative cells expressed increased levels of EGFR and were MAPK-signaling dependent, while slowly proliferating cells had higher levels of ErbB3 and p27, and are PI3K-dependent. The revelation of such molecular-phenotypic relationships validates the biological relevance of the largely phenomenological model of cell growth regulation disclosed herein.

To assess whether these correlations were predictive, a logistic regression model was developed to predict Pathway Bias of each call by input features (protein expression, PI3K pathway mutational status, or cellular properties of proliferation rate and tissue origin). Using basal protein expression as input features resulted in the best predictive value, as assessed by leave-one-out cross validation, producing 100% classification accuracy of cells into PI3K vs. MAPK-dependent categories (FIG. 2B). Notably, the Bias of the four cell lines that switch between PI3K and MAPK-dependence was accurately predicted by protein expression changes, and predicted values were intermediate between the solely PI3K- and MAPK-dependent categories, i.e., the Switching class of cells had lower predicted Bias than PI3K-dependent cells in the absence of heregulin (average 0.69 vs. 0.97, P=0.05; t-test), and higher predicted Bias than MAPK-dependent cells in the presence of heregulin (average −0.87 vs. −0.98, P=10−4; t-test). This class of cells appears to be molecularly poised between dependence on the two pathways, thus amenable to switching dependence in response to environmental stimuli (e.g., heregulin).

To determine whether this set of four protein biomarkers (EGFR, ErbB2, ErbB3, and CDKN1B) could be used to predict PI3K- vs. MAPK-bias in an independent data set. An internal ELISA-based protein profiling dataset of ErbB receptors (but not CDKN1B) was first searched for overlap with the Genomics of Drug Sensitivity in Cancer (GDSC) database, which consists of 714 cell lines screened for sensitivity to 138 cancer drugs. Of the 66 cell lines that exhibited an overlap, six of the HER2HI cells were predicted to be PI3K-biased (EGFRLO ERBB3HI) vs. eight that were predicted to be MAPK-biased (EGFRHIErbB3LO). Of the 138 cancer drugs, 13 were found to display differential activities between the groups (FIG. 2C). These included AKT and MEK inhibitors, validating the biological relevance of these protein markers, but also other inhibitors targeting regulators of metabolism (mTOR, AMPK), cell cycle (microtubules), and stress response (HDAC, HSP70). Evaluation of EGFR vs. ErbB3 protein expression is therefore important for designing combination treatment strategies for treating HER2+ disease.

### Example 4

### In Silico Screening of PI3K vs. MAPK-Pathway Dependent Tumors for Multi-Agent Growth Responses

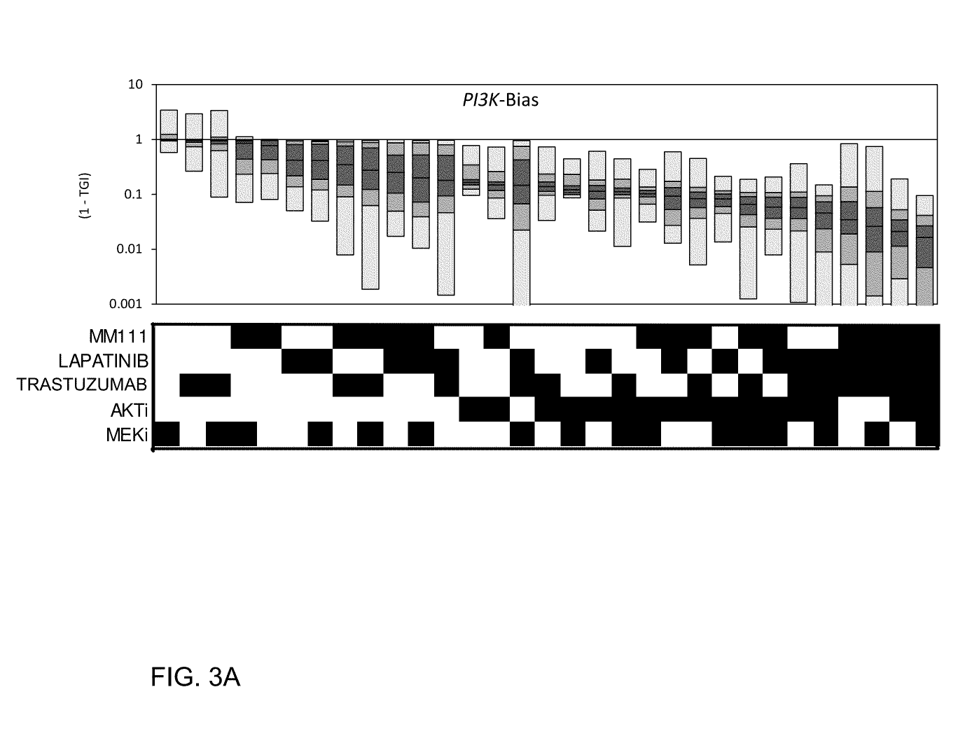

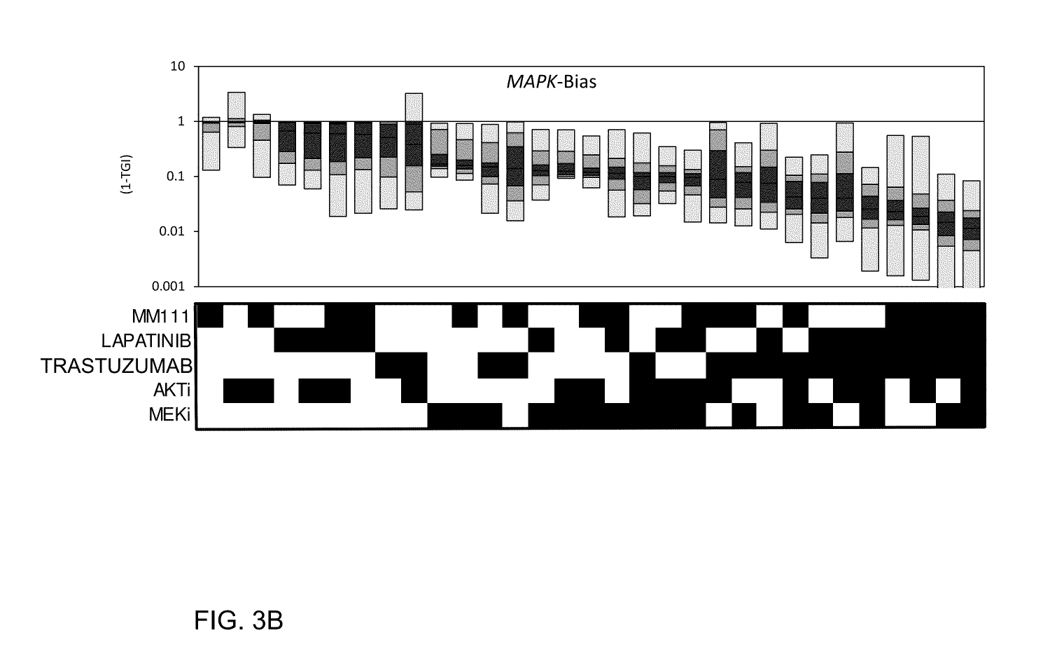

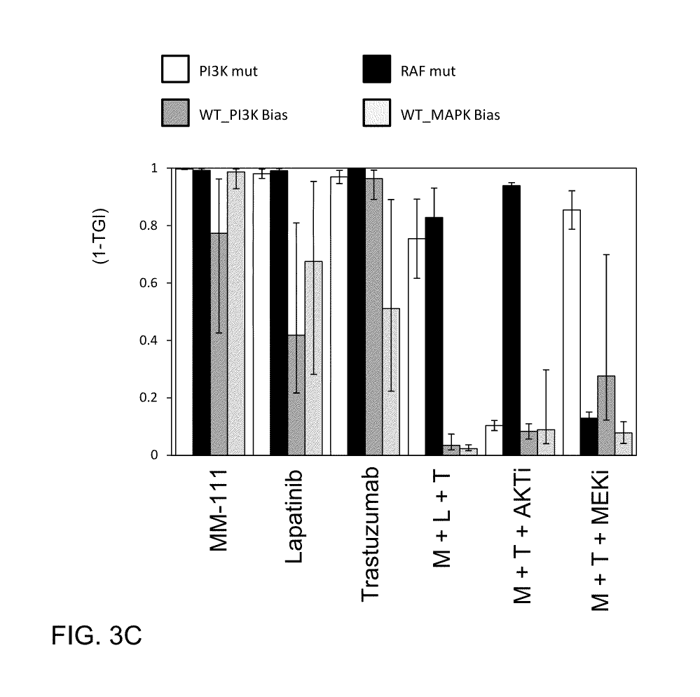

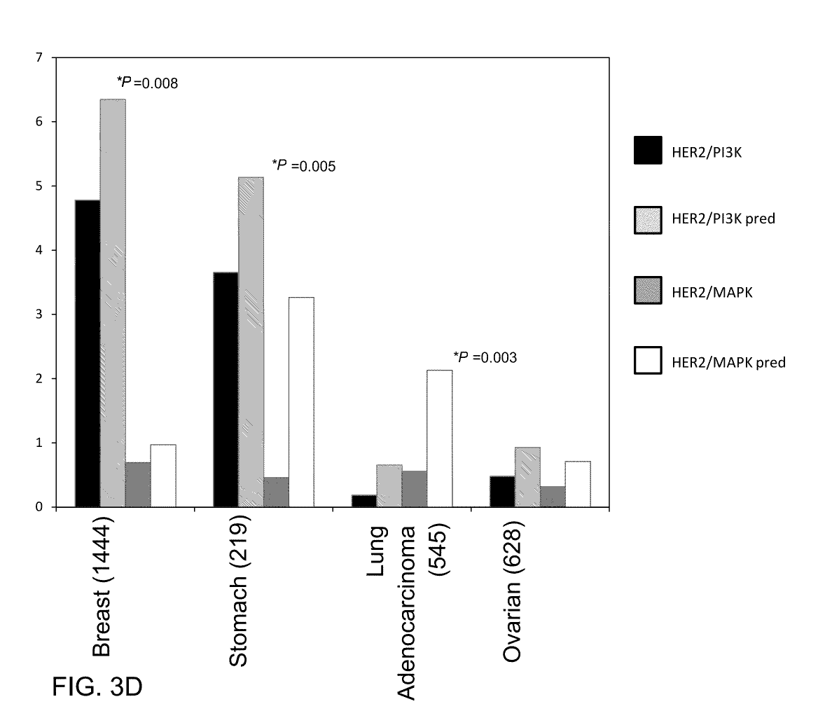

To determine how such PI3K vs. MAPK dependencies may affect responsiveness to combinations of clinically relevant targeted agents, in silico screening was performed. Five such agents were considered: the standard of care trastuzumab, a HER2-targted TKI (lapatinib), a HER3-targeted biologic (MM-111), and TKIs against the canonical PI3K/AKT and MAPK/ERK cascades: MK2206 (Merck, an AKT inhibitor) and trametinib (GSK1120212, a MEK inhibitor). A previously published computational model was used, which connects HER2-HER3 signaling, via PI3K/AKT and MAPK/ERK signaling cascades, to tumor growth, to assess all 32 possible combinations of the five agents. Ten protein and gene based putative biomarkers were randomly varied within biologically feasible ranges, and Monte Carlo simulations were employed to assess tumor growth responses across a synthetically heterogeneous population of tumors. Groups were separated for PI3K/AKT vs. MAPK/ERK signaling-dependent growth, and combinations rank ordered by median anti-tumor efficacy (FIG. 3A-B). Examining the contribution of individual agents, the AKTi containing regimens were determined to be significantly more effective in PI3K-dependent tumors (P=0.006; binomial test), while MEKi and trastuzumab-containing regimens were more effective in the MAPK-dependent tumors (P=0.1; binomial test). Regardless of pathway dependence, the combination of MM-111, trastuzumab, and lapatinib (MTL) was the most effective 3-drug regimen (ranked#4 overall in both cases).

Median responses, however, obscure the large variability (3 orders of magnitude) observed across the synthetic populations. To identify biomarkers predictive of MTL efficacy, parameter values (biomarkers) in the top 10% of non-responders vs. 50% of best responders to the combination were compared. The top predictors of resistance were PI3K- and MAPK-pathway-activating mutations in PI3K- and MAPK-dependent cells, respectively (P=2×10−16 and 3×10−15; Rank-sum test). High levels of heregulin were also predicted to confer additional relative resistance to the MTL in both contexts (P=3×10−4 and 1×10−4; Rank-sum test).

Combinations that most effectively treated the genetically defined HER2+ disease sub-populations (PI3K-mutant, MAPK-mutant, and dual-wild type) were then screened in silico. Simulations predicted that switching out lapatinib in the MTL combination for an AKT inhibitor or MEK inhibitor in the PI3K and MAPK mutant tumors, respectively, would be significantly more effective than the MTL triplet, or monotherapy with the agents in isolation (FIG. 3C). To assess the clinical relevance of these biomarkers in HER2+ cancer, mutational profiles for the genes ErbB2 (HER2), as well as commonly mutated components of the PI3K (PIK3CA, PIK3R1, and PTEN) and MAPK cascades (BRAF, KRAS, HRAS, NRAS) were extracted from primary tumor genome sequences represented in The Cancer Genome Atlas (TCGA). Samples harboring functional mutations in any of the three PI3K or MAPK pathway genes were annotated as PI3K and MAPK mutant, respectively.

Consistent with functional pathway dependencies of the cell lines under study, breast cancers had many more PI3K than MAPK pathway mutations, while the other indications (stomach, lung, and ovarian) either had more balanced mutational profiles (stomach, ovarian) or were MAPK-enriched (lung). Mutual exclusivity of genetic mutations within the same tumor is thought to be indicative of the genes mapping onto the same functional pathway. Based on this principle, the PI3K vs MAPK dependence in primary HER2+ cancers was estimated based on the frequency of co-occurrence of PI3K or MAPK pathway alterations with HER2 amplifications across the four indications (FIG. 3D). HER2-PI3K pathway co-occurrence was significantly less frequent than expected in breast tumors, while HER2-MAPK pathway co-occurrence was less than expected in stomach and lung tumors (Binomial test, P<0.01). Consistent with results from the functional profiling study described above, this suggests that HER2+ breast cancers are more likely to depend on PI3K signaling, while HER2+ lung and stomach cancer cells are more likely MAPK pathway-dependent. Furthermore, as the primary tumors represented in TCGA are largely untreated tissue resections, this suggests PI3K pathway activating mutations would be a more common mechanism of resistance to HER2 therapies in breast cancer, while MAPK-activation mutations a more common mechanisms of resistance in lung and stomach cancers. These results show that in second line HER2+ disease, refractory breast cancers are more likely to respond to PI3K pathway inhibitors, while lung and stomach cancers are more likely to respond to MAPK pathway inhibitors.

### Example 5

### PI3K and MAPK Activating Mutations Confer Resistance to HER2/HERS Inhibitor Combinations In Vitro

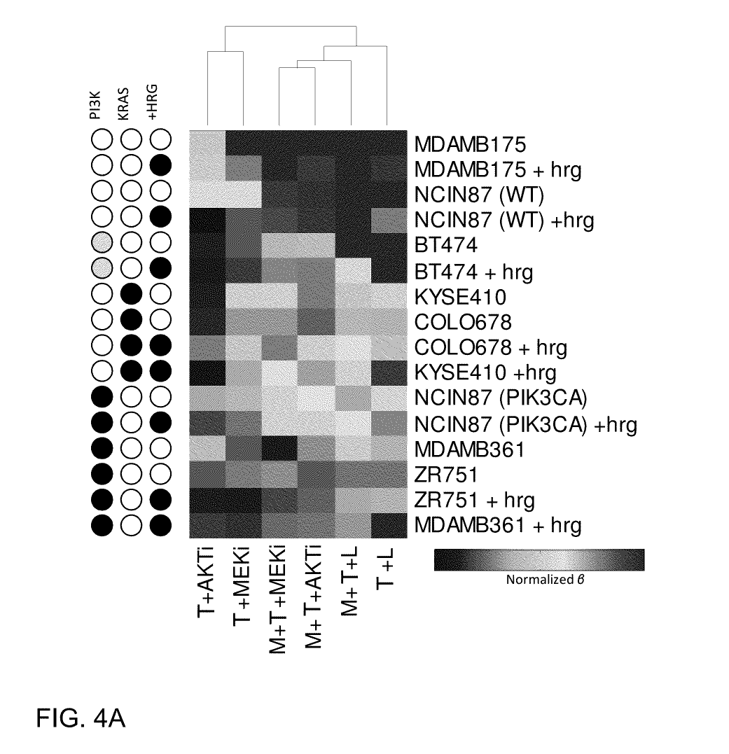

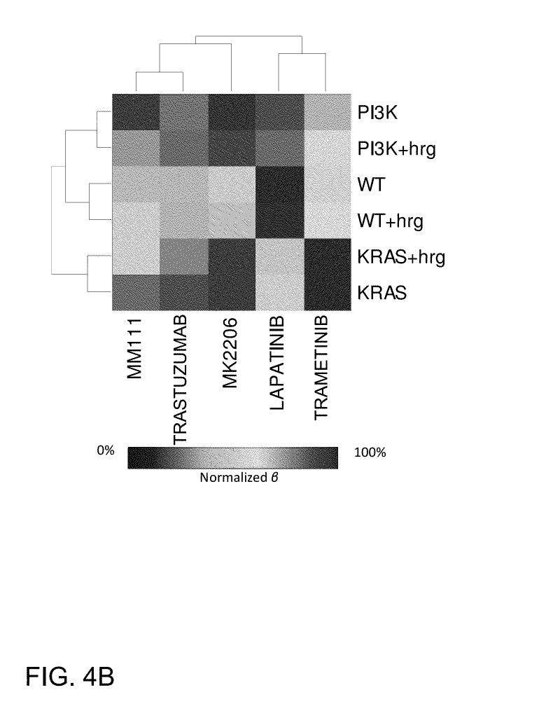

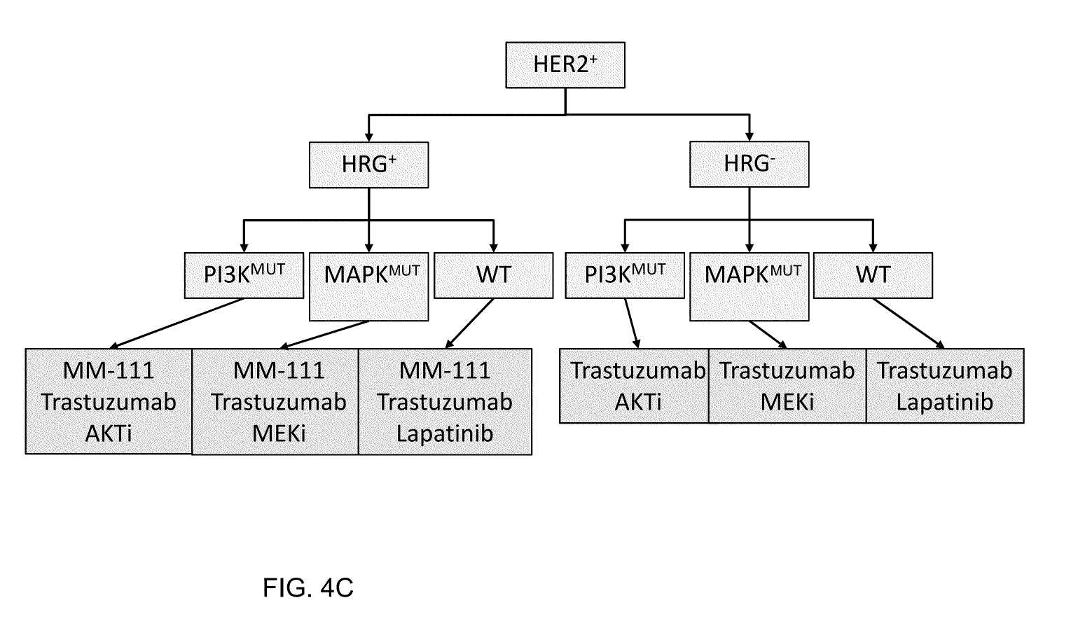

To assess the foregoing computational predictions experimentally, in vitro proliferative responses to MM-111, trastuzumab, and lapatinib combination treatments (MTL) were tested in eight HER2+ cell lines, as well as AKT and MEK inhibitor combinations, in the presence and absence of 5 nM exogenous HRG. This panel included cells harboring PI3KCA-activating mutations (both natural and engineered), PTEN deletions, and KRAS-activating mutations, as well as PI3K and MAPK-wild type cells. The results of these experiments are shown in FIG. 4A. To clarify and summarize the drug-response relationships, multivariate regression models were parameterized for each cell line, simulating % cell growth inhibition as linear combinations of the five input drug concentrations. Regression coefficients for each drug were grouped by mutational background and the presence or absence of heregulin stimulation, and average values computed (FIG. 4B).

These results show that the presence of HRG desensitizes cells to trastuzumab and lapatinib, and increases sensitivity to MM-111, regardless of genetic background. KRAS-mutant cells (KYSE-410 and COLO-678) are most sensitive to MEK inhibitor (trametinib) containing regimens, PI3K-mutant cells (MDA-MB-361, ZR-75-1, and NCI-N87-PIK3CA) are most sensitive to the AKT-inhibitor MK2206-containing regimens, and wild-type cells are most sensitive to lapatinib-containing regimens.

These data demonstrate that activating mutations within the PI3K and MAPK cascades are capable of mediating resistance to combinations of HER2/HER3 inhibitors in HER2+ cancers, and can be overcome by inclusion of AKT and MEK inhibitors in combination regimens. Trastuzumab (rather than lapatinib) is standard of care in HER2+ disease, and MM-111 overcomes HRG-mediated resistance to HER2-targeted therapy. The decision tree shown in FIG. 4C summarizes the patient selection methods suggested by these results. While specific assays and quantitative thresholds are not explicitly derivable from these data, this strategy is useful for designing precision drug combinations to treat HER2+ disease.

1. A method of selecting appropriate therapy for, or optimizing therapeutic efficacy of treatment of, a patient having a cancer, the method comprising:
(a) obtaining three or more biomarker readings obtained from a biological sample of the cancer from the patient, wherein the three or more biomarker readings include readings of levels of 1) HER2 and 2) heregulin (HRG) and of 3) mutational status of one or more genes selected from phosphoinositol-3-kinase pathway genes PIK3CA, PIK3R1 and PTEN, and mitogen-activated protein kinase (MAPK) pathway genes BRAF, KRAS, HRAS and NRAS; and
(b) (i) if the one or more readings indicate that the cancer is HER2+ and heregulin+ and that the mutational status of each of the one or more genes is wild-type, then having an effective amount of MM-111 administered to the patient in combination with administration of an effective amount of each of trastuzumab and a receptor tyrosine kinase inhibitor; or
(ii) if the one or more readings indicate that the cancer is HER2+ and heregulin+ and that the mutational status of a gene in the phosphoinositol-3-kinase pathway is mutant, then having an effective amount of MM-111 administered to the patient in combination with administration of an effective amount of each of trastuzumab and a mitogen-activated protein kinase kinase (MEK) inhibitor; or
(iii) if the one or more readings indicate that the cancer is HER2+ and heregulin+ and that the mutational status of a gene in the mitogen activated protein kinase pathway is mutant, then having an effective amount of MM-111 administered to the patient in combination with administration of an effective amount of each of trastuzumab and a protein kinase B (AKT) inhibitor.

2. The method of claim 1, wherein the mutational status of a gene in the PI3K pathway is mutant, and the gene is PIK3CA.

3. The method of claim 1, wherein the mutational status of a gene in the mitogen activated protein kinase pathway is mutant, and the gene is KRAS.

4. The method of claim 1, wherein the levels of HER2 are determined by IHC, and wherein if HER2 has a score of 2+ or 3+ by IHC, the biological sample is determined to be HER2+.

5. The method of claim 1, wherein the levels of HER2 are determined by the average number of receptors per cell, and wherein the average number of receptors per cell is 200,000 or greater.

6. The method of any one of the preceding claims, wherein the heregulin score represents a measure of RNA detected using one or more nucleic acid probes specific for heregulin.

7. The method of claim 6, wherein the RNA is detected by in situ hybridization using one or more nucleic acid probes that specifically hybridize to a nucleic acid comprising the heregulin sequence.

8. The method of any one of claims 1-5, wherein the heregulin score represents a measure of nucleic acid detected by RT-PCR using one or more nucleic acid probes that specifically hybridize to a nucleic acid comprising the heregulin sequence.

9. The method of any of the preceding claims, wherein the three or more readings is nine readings.

10. The method of claim 2, wherein the protein kinase B inhibitor is MK2206.

11. The method of claim 3, wherein the MEK inhibitor is trametinib.

12. The method of any one of the preceding claims, wherein the cancer is selected from the group consisting of ovarian cancer, breast cancer, uterine cancer, gastric cancer, stomach cancer, gastroesophageal junction cancer, esophageal cancer, and head and neck cancer.

13. The method of any one of the preceding claims, wherein the sample is a microtome section of a biopsy.

14. The method of any one of the preceding claims, wherein the sample is a microtome section of a formalin-fixed and paraffin embedded biopsy.

15. The method of claim 13 or 14, wherein the biopsy was obtained within 90 days prior to treating the patient.

16. The method of any one of claims 1 to 15, wherein the treatment produces at least one therapeutic effect selected from the group consisting of reduction in size of a tumor, reduction in the number of metastatic lesions over time, complete response, partial response, stable disease, increase in overall response rate, or a pathologic complete response.
